(19) United States
(12) Patent Application Publication
(10) Pub. No.: US 2024/0379216 A1
(43) Pub. Date: Nov. 14, 2024

(54) MEDICAL IMAGE PROCESSING APPARATUS, MEDICAL IMAGE PROCESSING METHOD, AND MEMORY MEDIUM

(71) Applicant: CANON MEDICAL SYSTEMS CORPORATION, Otawara-shi (JP)

(72) Inventor: Hideaki ISHII, Nasushiobara (JP)

(73) Assignee: CANON MEDICAL SYSTEMS CORPORATION, Otawara-shi (JP)

(21) Appl. No.: 18/658,365

(22) Filed: May 8, 2024

(30) Foreign Application Priority Data
May 10, 2023 (JP) 2023-077961

(51) Int. Cl. G16H 30/40; G06V 10/44

(52) U.S. Cl. CPC G16H 30/40; G06V 10/44

(57) ABSTRACT

A medical image processing apparatus according to an embodiment includes a processing circuitry that: obtains a first-type medical image, and obtains a second-type medical image which is taken at a different timing than the first-type medical image; extracts first-type feature points from the first-type medical image, and extracts second-type feature points from the second-type medical image; associates first-type feature points and second-type feature points; receives a correction input that, from among the pairs of the first-type feature point and the second-type feature point associated, is meant for associating the concerned first-type feature point with a different second-type feature point; calculates the position difference between the pre-correction position and the post-correction position of the second-type feature point captured in the second-type medical image and specified in the correction input; and based on the position difference, corrects the result of position adjustment between the first-type medical image and the second-type medical image.

### CROSS-REFERENCE TO RELATED APPLICATIONS

This application is based upon and claims the benefit of priority from Japanese Patent Application No. 2023-077961, filed on May 10, 2023, the entire contents of which are incorporated herein by reference.

### FIELD

Embodiments described herein relate generally to a medical image processing apparatus, a medical image processing method, and a memory medium.

### BACKGROUND

Conventionally, a healthcare professional such as an imaging diagnostician or a radiologist performs radiogram interpretation using a plurality of medical images captured using a medical diagnostic imaging apparatus. Moreover, regarding a tumor that requires follow-up examination, in the case of performing radiogram interpretation, the user who is a healthcare professional associates the position of the tumor in a medical image capturing the present state of the test subject with the position of the same tumor in a medical image capturing a former state of that test subject, and then performs radiogram interpretation.

### BRIEF DESCRIPTION OF THE DRAWINGS

FIG. 1 is a block diagram illustrating an exemplary configuration of a medical image processing system that includes a medical image processing apparatus according to an embodiment;

FIG. 2 is a block diagram illustrating an exemplary configuration of the medical image processing apparatus according to the embodiment;

FIG. 3 is a diagram for explaining an example of a display operation for displaying the association result regarding the disease sites according to the embodiment;

FIG. 4 is a diagram for explaining an example of the operation of receiving a correction input meant for correction in the association result of the disease sites according to the embodiment;

FIG. 5 is a diagram for explaining an example of the operation of presenting the candidate pairs for correction meant for correction in the association result of the disease sites according to the embodiment;

FIG. 6 is a flowchart for explaining an example of the operations performed in the medical image processing apparatus according to the embodiment;

FIG. 7 is a diagram illustrating an example of the display operation for displaying the association result of the disease sites according to a modification example;

FIG. 8 is a diagram for explaining an example of the operation of receiving a correction input meant for correction in the association result of the disease sites according to the modification example; and

FIG. 9 is a diagram for explaining an example of the operation of presenting the candidate pairs for correction meant for correction in the association result of the disease sites according to the modification example.

### DETAILED DESCRIPTION

A medical image processing apparatus according to an embodiment includes a processing circuitry that: obtains a first-type medical image, and obtains a second-type medical image which is taken at a different timing than the first-type medical image and which includes a region captured in the first-type medical image; extracts first-type feature points from the first-type medical image, and extracts second-type feature points from the second-type medical image; based on the result of position adjustment between the first-type medical image and the second-type medical image, associates first-type feature points and second-type feature points captured in the corresponding part in the first-type medical image and in the second-type medical image; receives a correction input that, from among the pairs of the first-type feature point and the second-type feature point associated with each other, is meant for associating the concerned first-type feature point with a different second-type feature point; calculates the position difference between the pre-correction position and the post-correction position of the second-type feature point captured in the second-type medical image and specified in the correction input; and based on the position difference, corrects the result of position adjustment between the first-type medical image and the second-type medical image.

An exemplary embodiment of a medical image processing apparatus, a medical image processing method, and a memory medium is described below in detail with reference to the accompanying drawings. The following explanation is given with reference to a medical image processing system that includes a medical image processing apparatus.

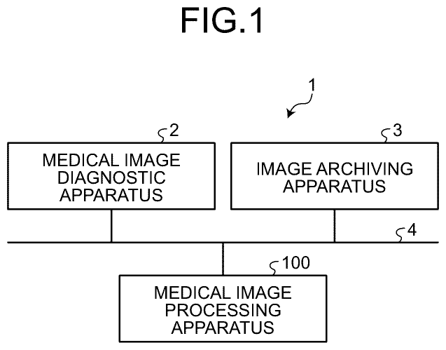

FIG. 1 is a diagram illustrating an exemplary configuration of a medical image processing system 1 that includes a medical image processing apparatus 100 according to the embodiment. The medical image processing system 1 illustrated in FIG. 1 includes the medical image processing apparatus 100, a medical image diagnostic apparatus 2, and an image archiving apparatus 3.

The medical image processing apparatus 100 is connected to the medical image diagnostic apparatus 2 and the image archiving apparatus 3 via, for example, a network 4 such as an in-hospital LAN (which stands for Local Area Network) installed in the concerned hospital. Herein, the apparatuses are capable of communicating with each other either directly or indirectly. For example, when the medical image processing system 1 is equipped with the PACS (which stands for Picture Archiving and Communication System), the apparatuses send and receive images among themselves according to the DICOM standard (DICOM stands for Digital Imaging and Communications in Medicine).

The medical image diagnostic apparatus 2 generates/collects medical images. For example, the medical image diagnostic apparatus 2 is an X-ray CT apparatus (CT stands for Computed Tomography) or an X-ray diagnostic apparatus.

Alternatively, the medical image diagnostic apparatus 2 can be an MRI apparatus (MRI stands for Magnetic Resonance Imaging), or an ultrasound diagnostic apparatus, or a SPECT apparatus (SPECT stands for Single Photon Emission Computed Tomography), or a PET apparatus (PET stands for Positron Emission computed Tomography). Still alternatively, the medical image diagnostic apparatus 2 can be a SPECT-CT apparatus configured by integrating a SPECT apparatus and an X-ray CT apparatus, or can be a PET-CT apparatus configured by integrating a PET apparatus or an X-ray CT apparatus, or can be a group of apparatuses mentioned above.

The medical image diagnostic apparatus 2 is capable of generating two-dimensional medical images, three-dimensional medical images (volume data), two-dimensional medical images arranged in chronological order, or three-dimensional medical images arranged in chronological order.

The medical image diagnostic apparatus 2 performs imaging of the test subject and collects medical images. For example, in an X-ray CT apparatus representing an example of the medical image diagnostic apparatus 2, an X-ray tube and an X-ray detector rotate with respect to the test subject, so that the X-rays that have passed through the test subject are detected and projection data is collected.

Then, in the X-ray CT apparatus, based on the collected projected data, various types of medical images are generated, such as two-dimensional CT images, three-dimensional CT images (volume data), two-dimensional CT images arranged in chronological order, or three-dimensional CT images arranged in chronological order.

Moreover, in the X-ray CT apparatus, based on the collected projection data, a plurality of two-dimensional CT images can be generated along a predetermined direction. For example, in the X-ray CT apparatus, two-dimensional CT images of a plurality of axial cross-sections can be generated along the body axis.

Then, the medical image diagnostic apparatus 2 sends the generated medical images to the image archiving apparatus 3. At the time of sending the medical images to the image archiving apparatus 3, the medical image diagnostic apparatus 2 also sends, for example, the following supplementary information: a patient ID enabling identification of the patient, an examination ID enabling identification of the examination, an apparatus ID enabling identification of the medical image diagnostic apparatus 2, and a series ID enabling identification of a single iteration of imaging performed by the medical image diagnostic apparatus 2.

The image archiving apparatus 3 represents a database for archiving medical images. More particularly, the image archiving apparatus 3 includes a memory in which the medical images sent by the medical image diagnostic apparatus 2 are stored for archival purpose.

The memory of the image archiving apparatus 3 is, for example, a semiconductor memory such as a random access memory (RAM) or a flash memory, or a storage such as a hard disk or an optical disc. In the image archiving apparatus 3, the medical images are archived in a corresponding manner to the patient IDs, the examination IDs, the apparatus IDs, and the series IDs. Thus, by performing a search using a patient ID, an examination ID, a device ID, or a series ID; the medical image processing apparatus 100 becomes able to obtain the required medical images from the image archiving apparatus 3.

The medical image processing apparatus 100 performs image processing with respect to medical images. The medical image processing apparatus 100 can be one of various types of apparatuses such as a workstation, a PACS image server or a PACS image viewer (PACS stands for Picture Archiving and Communication System), or an electronic health record system.

The medical image processing apparatus 100 performs a variety of processing with respect to the medical images obtained from the medical image diagnostic apparatus 2 or the image archiving apparatus 3. In the embodiment, the medical image processing apparatus 100 is used by a healthcare professional such as an imaging diagnostician or a radiologist to perform radiogram interpretation of medical images.

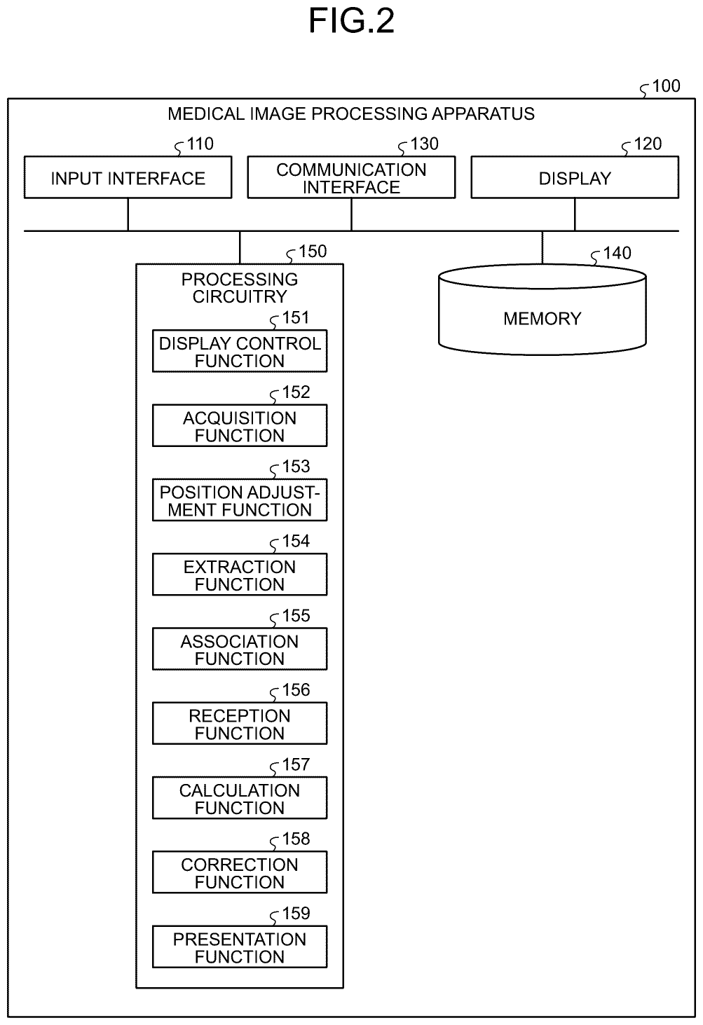

FIG. 2 is a block diagram illustrating an exemplary configuration of the medical image processing apparatus 100 according to the embodiment. As illustrated in FIG. 2, the medical image processing apparatus 100 includes an input interface 110, a display 120, a communication interface 130, a memory 140, and a processing circuitry 150.

The input interface 110 includes a pointing device, such as a mouse, and a keyboard for receiving input of various operations from a doctor, who represents the user, with respect to the medical image processing apparatus 100; and then transfers the instructions and the setting information, which are received from the user, to the processing circuitry 150.

The display 120 is a monitor that is viewed by the user. Under the control performed by the processing circuitry 150, the display 120 is used to display images to the user and to display a graphical user interface (GUI) for receiving various instructions and a variety of setting from the user via the input interface 110. The display 120 represents an example of a display unit. The communication interface 130 is a network interface card (NIC) that performs communication with other devices.

The memory 140 is, for example, a semiconductor memory such as a RAM or a flash memory, or a storage such as a hard disk or an optical disc. The memory 140 is used to store the medical images obtained from the medical image diagnostic apparatus 2 or the image archiving apparatus 3.

The processing circuitry 150 controls the constituent elements of the medical image processing apparatus 100. For example, as illustrated in FIG. 2, the processing circuitry 150 implements a display control function 151, an acquisition function 152, a position adjustment function 153, an extraction function 154, an association function 155, a reception function 156, a calculation function 157, a correction function 158, and a presentation function 159.

The processing functions such as the display control function 151, the acquisition function 152, the position adjustment function 153, the extraction function 154, the association function 155, the reception function 156, the calculation function 157, the correction function 158, and the presentation function 159 that represent the constituent elements of the processing circuitry 150 are stored as computer-executable programs in the memory 140.

The processing circuitry 150 is a processor that reads the computer programs from the memory 140 and executes them to implement the corresponding functions. In other words, after having read the computer programs, the processing circuitry 150 gets equipped with the functions illustrated in the processing circuitry 150 in FIG. 2.

The term “processor” mentioned above implies, for example, a central processing unit (CPU), a graphics processing unit (GPU), or an application specific integrated circuit (ASIC).

Moreover, the term “processor” implies a circuitry such as a programmable logic device. Examples of a programmable logic device include a simple programmable logic device (SPLD) and a complex programmable logic device (CPLD).

Another example of a programmable logic device is a field programmable gate array (FPGA). For example, when the processor is a CPU, it reads the computer programs stored in the memory 140 and executes them to implement the functions. On the other hand, for example, when the processor is an ASIC, instead of storing the computer programs in the memory 140, they are directly embedded into the circuitry of the processor.

Meanwhile, a processor according to the embodiment is not limited to be configurated as a singular circuitry. Alternatively, a plurality of independent circuitry can be combined to form a single processor in which the functions can be implemented. Still alternatively, a plurality of constituent elements illustrated in FIG. 2 can be integrated into a single processor in which the functions can be implemented.

Till now, the explanation was given about the overall configuration of the medical image processing system 1 that includes the medical image processing apparatus 100 according to the embodiment. With such a configuration, the medical image processing apparatus 100 supports efficient position adjustment with respect to medical images. More particularly, the processing circuitry 150 of the medical image processing apparatus 100 includes the display control function 151, the acquisition function 152, the position adjustment function 153, the extraction function 154, the association function 155, the reception function 156, the calculation function 157, the correction function 158, and the presentation function 159 as the function units, and performs the following operations.

The display control function 151 performs control for displaying a variety of information in a display device. For example, the display control function 151 displays medical images and a GUI in the display 120. Regarding a variety of information that is displayed by the display control function 151 in the display 120, the explanation is given later.

The acquisition function 152 obtains medical images. For example, using the patient ID of the test subject who is to be examined, the acquisition function 152 obtains, from the medical image diagnostic apparatus 2 or the image archiving apparatus 3, a medical image in which the present state of the test subject is captured (hereinafter, also referred to as a present medical image). Herein, a present medical image represents an example of a first-type medical image. Meanwhile, a present medical image can be an image in which the recent state of the test subject is captured. A present medical image includes a region that indicates the state of the target body part for examination of the test subject.

In an identical manner, the acquisition function 152 also obtains a medical image of the test subject that is taken at an earlier timing than a present medical image (hereinafter, also referred to as a former medical image) and that is to be associated with a present medical image. In this case, a former medical image represents an example of a second-type medical image. A former medical image includes a region that indicates the target body part for examination of the test subject.

The position adjustment function 153 performs position adjustment with respect to a particular medical image and another medical image that is taken at a different timing than the particular medical image. For example, the position adjustment function 153 performs position adjustment with respect to a present medical image and a former medical image.

As an example, the position adjustment function 153 extracts one or more anatomic landmarks from a present medical image and a former medical image, and superimposes the anatomic landmarks of the present medical image and the anatomic landmarks of the former medical image, so as to perform position adjustment with respect to the present medical image and the former medical image. If the liver represents the target body part, then the position adjustment function 153 treats the anatomic feature points of the liver, which are based on the morphological features, as the anatomic landmarks and accordingly performs position adjustment.

Meanwhile, the position adjustment function 153 can refer to the segmentation result obtained in regard to the present medical image and the former medical image according to a known segmentation technology, and can accordingly perform position adjustment with respect to the present medical image and the former medical image.

The extraction function 154 extracts disease sites from medical images. For example, the extraction function 154 extracts disease sites from a present medical image and a former medical image. In that case, a disease site captured in a present medical image represents an example of a first-type feature point, a disease site captured in a former medical image represents an example of a second-type feature point. Examples of a disease site include a tumor occurring in the test subject. Meanwhile, in order to extract disease sites, a known technology can be implemented. Herein, the extraction function 154 implements the same method for extracting disease sites from a present medical image and from a former medical image.

As an example, the extraction function 154 extracts disease sites from a medical image, which is input thereto, using an already-learnt model equipped with the function of deducing the information indicating the disease sites in the medical image (i.e., the information indicating the position and the shape of each disease site in the medical image). In that case, the already-learnt model is obtained as a result of performing learning with the use of a known method of machine learning or deep learning and by treating a medical image as the input-side teacher data and by treating the information indicating the disease sites as the output-side teacher data.

Meanwhile, regarding a former medical image, the extraction result regarding the disease sites can be stored in a memory system such as the image archiving apparatus 3, and the acquisition function 152 can obtain that medical image along with obtaining the extraction result regarding the disease sites in the medical image.

The association function 155 associates the disease sites captured in a particular medical image with the disease sites captured in another medical image other than the particular medical image. For example, the association function 155 implements the block matching method and associates the positions of the disease sites, which are captured in a present medical image and extracted by the extraction function 154, with the positions of the disease sites captured in a former medical image.

In that case, for example, regarding each region representing a disease site captured in a present medical image; the association function 155 detects, from among the regions representing the disease sites captured in a former medical image, such a subregion which has the degree of coincidence to be equal to or greater than a certain level with respect to a subregion including specific pixels and their surrounding pixels in the concerned region captured in the present medical image.

Herein, examples of the method for calculating the degree of coincidence include the SAD method (SAD stands for Sum of Absolute Difference) and the SSD method (SSD stands for Sum of Squared Difference). In the SAD method, the association function 155 takes the absolute values of the pixel-by-pixel differences between a subregion captured in the present medical image and a subregion captured in the former medical image, and treats the sum of the absolute values as the evaluation value of the degree of coincidence. Herein, smaller the evaluation value, the higher becomes the degree of coincidence.

Alternatively, in the SAD method, the association function 155 takes the square values of the pixel-by-pixel differences between a subregion captured in the present medical image and a subregion captured in the former medical image, and treats the sum of the square values as the evaluation value of the degree of coincidence. Herein, smaller the evaluation value, the higher becomes the degree of coincidence.

If the position of the detected subregion in the former medical image is within a predetermined range of the position in the former medical image which corresponds to the position of the concerned subregion in the present medical image, then the association function 155 associates the disease site corresponding to the subregion captured in the present medical image with the disease site corresponding to the subregion captured in the former medical image.

The method for associating the disease sites as explained above is only exemplary. That is, the disease sites can be associated also using some other method other than the block matching method.

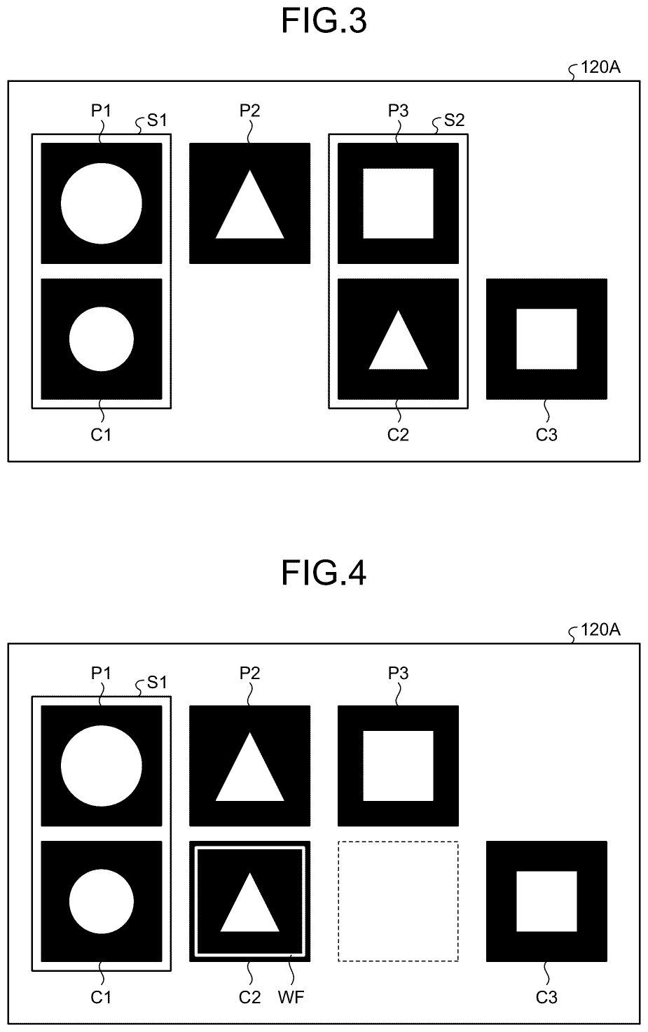

Subsequently, under the control performed by the display control function 151, the association result is displayed in the display 120. FIG. 3 is a diagram for explaining an example of a display operation for displaying the association result regarding the disease sites. In FIG. 3 is illustrated an example of a first screen 120A in which the association result regarding the disease sites is displayed.

In the example illustrated in FIG. 3, the first screen 120A is configured to have a two-tier display region. In the upper tier in FIG. 3 are illustrated disease sites P1 to P3 that are captured in a former medical image. In the lower tier in FIG. 3 are illustrated disease sites C1 to C3 that are captured in a present medical image. Herein, if the liver represents the target body part, then the disease sites P1 to P3 and the disease sites C1 to C3 represent tumors occurring in the liver of the test subject.

As an example, the disease sites P1 to P3 are displayed side by side in the horizontal direction in the order corresponding to the their positions in the former medical image which is taken along the body axis direction of the test subject. Regarding the disease sites C1 to C3 too, the display arrangement is same. Alternatively, the disease sites P1 to P3 and the disease sites C1 to C3 can be displayed in the order corresponding to their positions along the perpendicular direction to the body axis direction.

Meanwhile, FIG. 3 is only an exemplary diagram. Thus, regarding the disease sites captured in a present medical image and the disease sites captured in a former medical image, the number of displayed disease sites is not limited to three. Moreover, the number of disease sites captured in a present medical image can be different than the number of disease sites captured in a former medical image.

In the example illustrated in FIG. 3, the association result obtained by the association function 155 is illustrated in first-type display frames S (S1 and S2). In this case, the first-type display frames S represent examples of association information. The first-type display frame S1 indicates that the disease site P1 is associated with the disease site C1. The first-type display frame S2 indicates that the disease site P3 is associated with the disease site C2. Moreover, in the example illustrated in FIG. 3, the first screen 120A indicates that the disease sites P2 and C3 are not associated with any other disease sites.

In this way, as a result of displaying the correspondence relationship between the disease sites captured in a former medical image and the disease sites captured in a present medical image, it becomes possible for the user to easily figure out which disease sites captured in the present medical image are associated with which disease sites captured in the former medical image.

Meanwhile, in FIG. 3, although the correspondence relationship between the disease sites captured in the present medical image and the disease sites captured in the former medical image is illustrated in the first-type display frames S, the method for expressing the correspondence relationship is not limited to that method. Alternatively, for example, the disease sites captured in the present medical image can be joined by straight lines to the corresponding disease sites captured in the former medical image, so that the correspondence relationship is expressed. Still alternatively, for example, instead of displaying the first-type display frames S, the corresponding disease sites can be displayed either one above the other or side by side, so that the correspondence relationship is expressed.

Returning to the explanation with reference to FIG. 2, the reception function 156 receives a correction input meant for correction in the association regarding one of the pairs of disease sites that includes a disease site captured in a particular medical image and a disease site captured in another medical image other than the particular medical image. For example, the reception function 156 receives, from the user via the input interface 110, a correction input meant for associating a single disease site captured in a present medical image with a disease site captured in a former medical image.

FIG. 4 is a diagram for explaining an example of the operation of receiving a correction input meant for correction in the association result of the disease sites. In FIG. 4 is illustrated an exemplary case in which a correction input is received from the user via the first screen 120A illustrated in FIG. 3.

For example, the user uses a mouse to drag one of the disease sites C1 to C3, which are captured in the present medical image, below a disease site to be associated from among the disease sites P1 to P3 captured in the former medical image, and thus performs a correction input meant for correction in the association. In the example illustrated in FIG. 4, the reception function 156 receives a correction input in which the disease site C2, which has been associated with the disease site P3, is to be moved below the disease site P2 in order to associate the disease site C2 with the disease site P2.

Meanwhile, a white frame WF indicates the disease site in the present medical image for which a correction input is received. In the example illustrated in FIG. 4, the white frame WF indicates that the disease site C2 represents the disease site for which a correction input is received.

For example, when a disease site captured in the present medical image is selected as the target for a correction input, the white frame WF gets displayed at the selected disease site. At that time, if a first-type frame S is being displayed at that disease site, that first-type frame S gets deleted. In the example illustrated in FIG. 4, the first-type frame S2 in which the disease site P3 is associated with the disease site C2 as illustrated in FIG. 3 gets deleted, and instead the white frame WF is displayed at the disease site C2.

Returning to the explanation with reference to FIG. 2, the calculation function 157 calculates the difference between the pre-correction position and the post-correction position of the disease site for which the correction input was received. For example, the calculation function 157 calculates the difference between the pre-correction position and the post-correction position of the disease site, for which the correction input was received, in the former medical image.

As an example, the calculation function 157 calculates, as a vector, the difference between the following positions: the position of the median point of that disease site in the former medical image which was associated, before correction, with the disease site in the present medical image as specified in the correction input, and the position of the median point of that disease site in the former medical image which is associated, after correction, with the concerned disease site in the present medical image.

In the examples illustrated in FIGS. 3 and 4, the calculation function 157 calculates, as a vector, the difference between the median point of the disease site P3 that was associated, before correction, to the disease site C2 and the median point of the disease site P2 that is associated, after correction, to the disease site C2.

Meanwhile, the method explained above is only an example of the method for calculating the difference between the pre-correction position and the post-correction position of a disease site. That is, the method for calculating the difference between the positions is not limited to the method explained above.

The correction function 158 corrects the result of position adjustment regarding medical images. For example, based on the difference between the positions as calculated by the calculation function 157, the correction function 158 corrects the result of position adjustment between the present medical image and the former medical image as obtained by the position adjustment function 153.

As an example, in the state in which the position of the present medical image is fixed, the correction function 158 moves the former medical image by the distance and in the direction indicated by the vector calculated by the calculation function 157, and accordingly corrects the result of position adjustment as obtained by the position adjustment function 153.

Based on the corrected result of position adjustment, the presentation function 159 presents, from among one or more disease sites, candidate disease sites for correction for which the association targets are to be changed. For example, firstly, according to the corrected result of position adjustment as obtained by the correction function 158, the association function 155 again performs the association operation for associating the disease sites in the present medical image with the disease sites in the former medical image. In this case, the association function 155 can perform the association operation only regarding the disease sites other than the pairs of disease sites involved in the correction input.

The presentation function 159 compares the association result before and after the correction in the result of position adjustment, and detects pairs of disease sites in each of which a disease site captured in the present medical image and a disease site captured in the former medical image have a change in their association result. Then, the presentation function 159 presents those pairs as the candidate pairs for correction meant for correction in the association result of the disease sites. The candidate pairs for correction presented by the presentation function 159 are, for example, displayed in the display 120 under the control performed by the display control function 151.

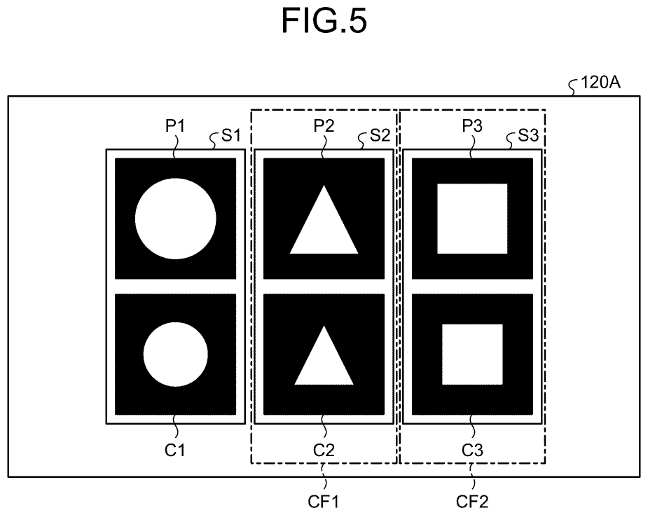

FIG. 5 is a diagram for explaining an example of the operation of presenting the candidate pairs for correction meant for correction in the association result of the disease sites. In FIG. 5 is illustrated an example in which the candidate pairs for correction are presented according to the correction input received from the user via the first screen 120A illustrated in FIG. 4.

In the first screen 120A illustrated in FIG. 5, the first-type display frame S1 indicates that the disease site P1 is associated with the disease site C1. Moreover, the first-type frame S2 indicates that the disease site P2 is associated with the disease site C2. Furthermore, a first-type frame S3 indicates that the disease site P3 is associated with the disease site C3.

Moreover, in the example illustrated in FIG. 5, second-type display frames CF (CF1 and CF2) indicate such pairs of disease sites in each of which a disease site captured in the present medical image and a disease site captured in the former medical image have had a change in their association result due to the correction in the result of position adjustment.

The second-type display frame CF1 indicates that the disease sites P2 and C2 that were not associated before the correction are now associated with each other due to the correction in the result of position adjustment. In an identical manner, the second-type display frame CF2 indicates that the disease sites P3 and C3 that were not associated before the correction are now associated with each other.

As a result, the user becomes able to easily figure out the pairs of disease sites in each of which a disease site captured in the former medical image and a disease site captured in the present medical image have a change in their association result.

In the example illustrated in FIG. 5, the pair of disease sites P1 and C1 that does not have any change in the combination before and after the correction is also displayed in the first screen 120A. However, that is not the only possible display form.

For example, in the case of presenting the candidates for correction in the association, the pairs of disease sites not having any change in the combination can be kept hidden in the first screen 120A. Alternatively, for example, as illustrated in FIG. 5, when the disease sites are displayed side by side in a predetermined order from the left side, only those pairs of disease sites which are positioned on the right side of the pair of disease sites involved in the correction input can be displayed. In that case, regarding the pairs of disease sites which are positioned on the left side of the pair of disease sites involved in the correction input, the association function 155 need not perform the association operation again.

Meanwhile, the reception function 156 can further receive input for correction in the association from the user via the first screen 120A illustrated in FIG. 5. In that case, the correction function 158 can further correct the result of position adjustment or can correct only the association result. As a result, even after correcting the result of position adjustment, if errors are still present in the association, then the user becomes able to easily correct the association.

If the association result is finalized when the reception function 156 receives, from the user via the first screen 120A, a confirmation input meant for confirmation of the association result, the processing circuitry 150 can send the association result to the image archiving apparatus 3 via the communication interface 130. In that case, in the image archiving apparatus 3, the association result is archived along with the concerned medical images.

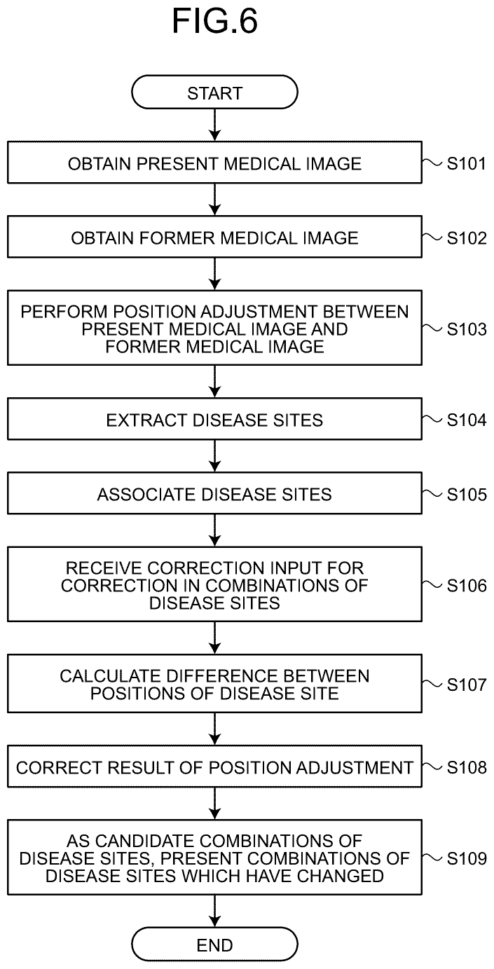

Given below is the explanation of the operations performed in the medical image processing apparatus 100 according to the embodiment. FIG. 6 is a flowchart for explaining an example of the operations performed in the medical image processing apparatus 100 according to the embodiment.

Firstly, the acquisition function 152 obtains a present medical image (Step S101). For example, using the patient ID of the test subject, the acquisition function 152 obtains, from the medical image diagnostic apparatus 2 or the image archiving apparatus 3, a medical image capturing the present state of the test subject (i.e., obtains a present medical image).

Then, the acquisition function 152 obtains a former medical image (Step S102). For example, in an identical manner, the acquisition function 152 obtains a medical image of the concerned patient that is taken at an earlier timing than the present medical image obtained at Step S101 (i.e., obtains a former medical image).

Subsequently, the position adjustment function 153 performs position adjustment between the present medical image and the former medical image (Step S103). For example, the position adjustment function 153 uses anatomic landmarks and performs position adjustment between the present medical image and the former medical image.

Then, the extraction function 154 extracts disease sites from the present medical image and the former medical image (Step S104). For example, the extraction function 154 obtains an already-learnt model by performing learning with the use of a known method of machine learning or deep learning, and extracts disease sites from the present medical image and the former medical image.

Then, the association function 155 associates the disease sites captured in the present medical image with the disease sites captured in the former medical image (Step S105). For example, the association function 155 implements the block matching method and associates the disease sites captured in the present medical image with the disease sites captured in the former medical image.

Subsequently, the reception function 156 receives a correction input meant for correction in the combinations of disease sites (Step S106). For example, from the user via the screen on which the combinations of disease sites are displayed, the reception function 156 receives a correction input meant for correction in a single combination of disease sites.

Then, the calculation function 157 calculates the difference between the pre-correction position and the post-correction position of the disease site, for which the correction input was received, in the former medical image (Step S107). For example, the calculation function 157 calculates, as a vector, the difference between the following positions: the position of the median point of that disease site in the former medical image which was associated, before correction, with the disease site in the present medical image as specified in the correction input, and the position of the median point of that disease site in the former medical image which is associated, after correction, with the concerned disease site in the present medical image.

Subsequently, the correction function 158 corrects the result of position adjustment (Step S108). For example, in the state in which the position of the present medical image is fixed, the correction function 158 moves the former medical image by the distance and in the direction indicated by the vector calculated at Step S107, and accordingly corrects the result of position adjustment as obtained at Step S103.

Then, the presentation function 159 presents the candidate combinations of disease sites for correction (Step S109). That marks the end of the operations. For example, based on the result of position adjustment as corrected at Step S108, the association function 155 again performs the association operation. After again performing the association operation, the presentation function 159 presents, as the candidate combinations of disease sites for correction, such combinations of disease sites which have changed after the correction as compared to the pre-correction state.

As explained above, in the medical image processing apparatus 100 according to the embodiment, a disease site captured in a present medical image and a disease site captured in a former medical image are associated according to the result of position adjustment between the present medical image and the former medical image. Moreover, in the medical image processing apparatus 100 according to the embodiment, a correction input meant for correction in the association of a disease site captured in the present medical image with a disease site captured in the former medical image is received from the user; the difference in the pre-correction position and the post-correction position of the disease site in the former medical image is calculated according to the details of the correction input; the result of position adjustment is corrected based on the difference in the positions; and the pairs of disease sites that have had a change in the combination according to the correction of the result of position adjustment are presented as the candidate pairs for correction.

As a result, the disease sites captured in the present medical image get automatically associated with the disease sites captured in the former medical image. Hence, at the time of performing radiogram interpretation, the user need not take into account the correspondence relationship between the disease sites captured in the present medical image and the disease sites captured in the former medical image. That is, the medical image processing apparatus 100 according to the embodiment can support the user in performing radiogram interpretation in an efficient manner.

Meanwhile, the position adjustment between a present medical image and a former medical image is not perfect. Hence, it is conceivable to have errors in the result of position adjustment. In that case, due to the errors in the result of position adjustment, the result of association of the disease sites captured in the present medical image with the disease sites captured in the former medical image may include errors. In such a case too, in the medical image processing apparatus 100, regarding a single combination of disease sites, a correction input is received from the user, so that the result of position adjustment can be corrected according to the details of the correction input. Thus, in the medical image processing apparatus 100 according to the embodiment, position adjustment between medical images taken at different timings can be performed in an efficient manner.

Moreover, in the medical image processing apparatus 100 according to the embodiment, since the association operation is again performed based on the corrected result of position adjustment, the candidate association results for correction can be presented to the user. Thus, even when there are errors in the association of a plurality of disease sites, merely by performing a correction input regarding one of the combinations of disease sites, the user becomes able to understand a plurality of candidate association results for correction.

Meanwhile, the embodiment described above can be appropriately modified by varying some of the configurations or the functions of the apparatuses included in the medical image processing system 1. Given below is the explanation of a modification example of the embodiment as another embodiment. In the following explanation, the focus is on explaining the differences with the embodiment described above. Thus, regarding the same details as the details already explained above, the detailed explanation is not given again. The modification example explained below can be implemented either individually or in combination.

### Modification Example

In the embodiment described above, the explanation is given about the case in which the disease sites captured in a present medical image and the disease sites captured in a former medical image are tiled horizontally as images illustrating only the disease sites. However, alternatively, the disease sites captured in a present medical image and the disease sites captured in a former medical image can be displayed in images illustrating the target body part in entirety.

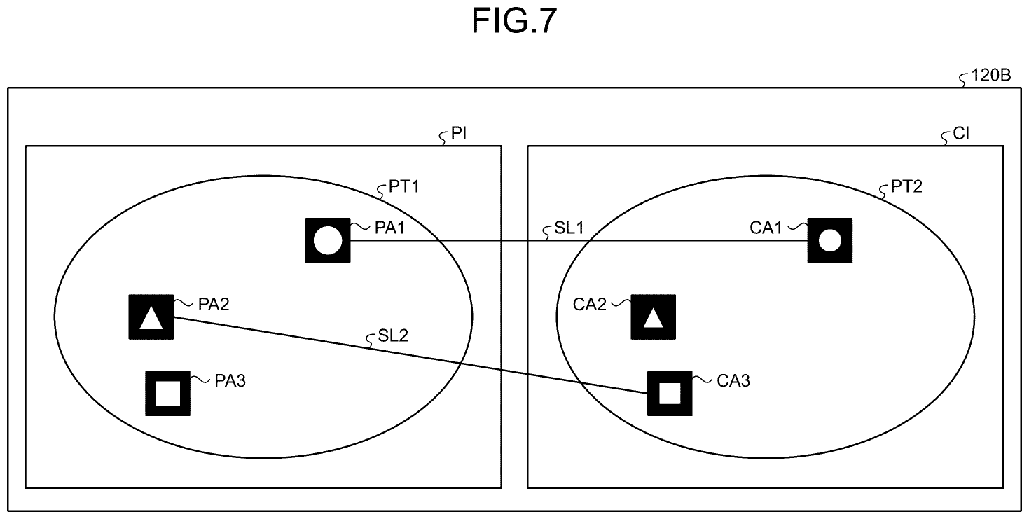

FIG. 7 is a diagram illustrating an example of the display operation for displaying the association result of the disease sites according to the modification example. In FIG. 7 is illustrated an example of a second screen 120B in which the association result of the disease sites is displayed along with displaying the target body part in entirety.

In the example illustrated in FIG. 7, the second screen 120B is configured with a pair of display regions arranged horizontally. In the left display region in FIG. 7 is illustrated a test subject image PT1 that includes disease sites PA1 to PA3 captured in a former medical image PI. In the right display region in FIG. 7 is displayed a test subject image PT2 that includes disease sites CA1 to CA3 captured in a present medical image CI. If the liver represents the target body part, then the test subject images PT1 and PT2 represent medical images enabling understanding of the whole picture of the liver.

Meanwhile, FIG. 7 is only an exemplary diagram. Thus, regarding the disease sites captured in a present medical image CI and the disease sites captured in a former medical image PI, the number of displayed disease sites is not limited to three. Moreover, the number of disease sites captured in a present medical image CI can be different than the number of disease sites captured in a former medical image PI.

Meanwhile, in the example illustrated in FIG. 7, the association result obtained by the association function 155 is illustrated using first-type straight lines SL (SL1 and SL2). The first-type straight line SL1 indicates that the disease site PA1 is associated with the disease site CA1. The first-type straight line SL2 indicates that the disease site PA2 is associated with the disease site CA3.

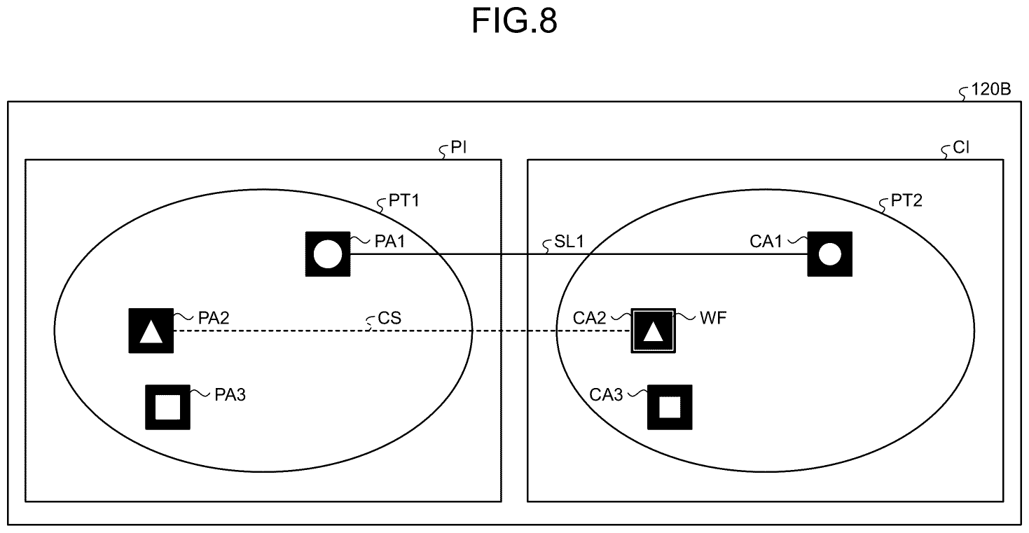

FIG. 8 is a diagram for explaining an example of the operation of receiving a correction input meant for correction in the association result of the disease sites according to the modification example. In FIG. 8 is illustrated an exemplary case in which a correction input is received from the user via the Second screen 120B illustrated in FIG. 7.

For example, the user uses a mouse to drag one of the disease sites CA1 to CA3, which are captured in the present medical image CI, within the predetermined range of a disease site to be associated from among the disease sites PA1 to PA3 captured in the former medical image PI, and thus performs a correction input meant for correction in the association. In the example illustrated in FIG. 8, the reception function 156 receives a correction input in which the disease site CA2, which is not associated with any other disease site, is to be moved within the predetermined range of the disease site PA2 in order to associate the disease site CA2 with the disease site PA2.

In an identical manner to the example illustrated in FIG. 4, the white frame WF indicates the disease site in the present medical image CI for which a correction input is received. In the example illustrated in FIG. 8, the white frame WF indicates that the disease site CA2 represents the disease site for which a correction input is received.

Herein, a second-type straight line CS indicates the combination of disease sites which includes a disease site captured in the present medical image CI and a disease site captured in the former medical image PI and for which a correction input is received. In the example illustrated in FIG. 8, the second-type straight line CS indicates that the disease sites PA2 and CA2 represent the pair of disease sites for which a correction input is received.

Moreover, for example, upon the selection of the disease site in the present medical image CI for which a correction input is received, the white frame WF gets displayed at the selected disease site. At that time, if a first-type straight line SL is being displayed at that disease site, that first-type straight line SL gets deleted.

In the example illustrated in FIG. 8, the first-type straight line SL2 which indicates that the disease site PA3 is associated with the disease site CA2 as illustrated in FIG. 7 gets deleted, and instead the white frame WF is displayed at the disease site CA2. Moreover, in the example illustrated in FIG. 8, instead of displaying the first-type straight line SL2, the second-type straight line CS is displayed to join the disease site PA2 to the disease site CA2.

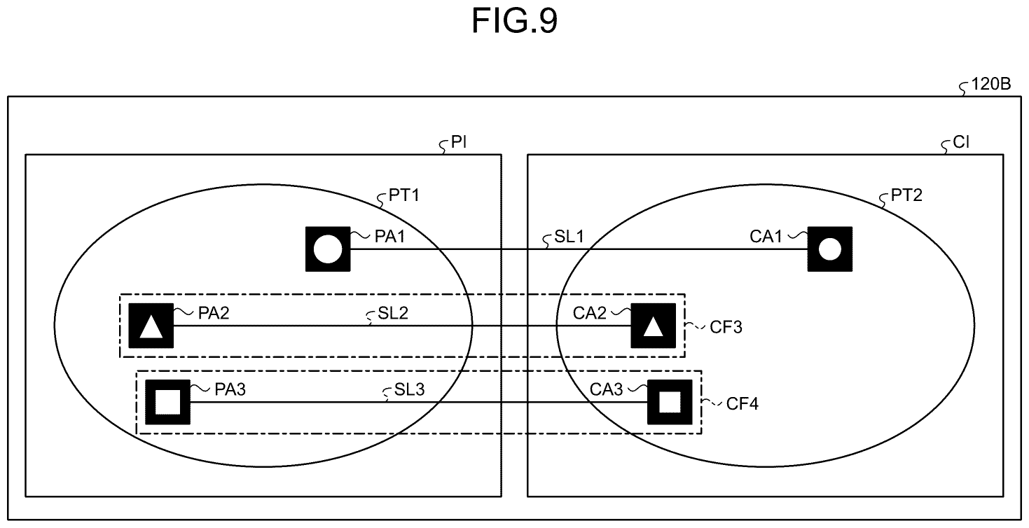

FIG. 9 is a diagram for explaining an example of the operation of presenting the candidate pairs for correction meant for correction in the association result of the disease sites according to the modification example. In FIG. 9 is illustrated an example in which the candidate pairs for correction in the association result are presented according to the correction input received from the user via the second screen 120B illustrated in FIG. 8.

In the second screen 120B illustrated in FIG. 9, the first-type straight line SL1 indicates that the disease site PA1 is associated with the disease site CA1. Moreover, the first-type straight line SL2 indicates that the disease site PA2 is associated with the disease site CA2. Furthermore, a first-type straight line SL3 indicates that the disease site PA3 is associated with the disease site CA3.

Moreover, in the example illustrated in FIG. 9, second-type display frames CF (CF3 and CF4) indicate such pairs of disease sites in each of which a disease site captured in the present medical image CI and a disease site captured in the former medical image PI have had a change in their association result due to the correction in the result of position adjustment.

The second-type display frame CF3 indicates that the disease sites PA2 and CA2 that were not associated before the correction are now associated with each other due to the correction in the result of position adjustment. In an identical manner, the second-type display frame CF4 indicates that the disease sites PA3 and CA3 that were not associated before the correction are now associated with each other.

According to the modification example, in the same screen, an image illustrating the target body part in entirety is displayed along with the correspondence relationship between the disease sites captured in a present medical image and the disease sites captured in a former medical image. That enables the user to understand, with more ease, the state of the disease sites in the test subject.

The constituent elements of the apparatuses illustrated in the drawings are merely conceptual, and need not be physically configured as illustrated. The constituent elements, as a whole or in part, can be separated or integrated either functionally or physically based on various types of loads or use conditions. Moreover, the process functions implemented in the apparatuses are entirely or partially implemented by a CPU or by computer programs that are analyzed and executed by a CPU, or are implemented as hardware by wired logic.

Meanwhile, the methods described in the embodiment can be implemented by executing a computer program written in advance in a computer such as a personal computer or a workstation. The program can be distributed over a network such as the Internet. Alternatively, the computer program can be stored in a computer-readable recording medium such as a hard disk, a flexible disk (FD), a compact disk read only memory (CD-ROM), a magneto-optical (MO) disk, or a digital versatile disk (DVD). Thus, a computer can read the computer program from the recording medium and execute it.

According to at least one of the embodiments described above, position adjustment between medical images taken at different timings can be performed in an efficient manner.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising a processing circuitry that
obtains a first-type medical image, and obtains a second-type medical image which is taken at a different timing than the first-type medical image and which includes a region captured in the first-type medical image,
extracts a first-type feature point from the first-type medical image, and extracts a second-type feature point from the second-type medical image,
based on result of position adjustment between the first-type medical image and the second-type medical image, associates a first-type feature point and a second-type feature point captured in corresponding part in the first-type medical image and in the second-type medical image,
receives a correction input that, from among pairs of the first-type feature point and the second-type feature point associated with each other, is meant for associating the first-type feature point with other second-type feature point,
calculates position difference between pre-correction position and post-correction position of second-type feature point captured in the second-type medical image and specified in the correction input, and
based on the position difference, corrects result of position adjustment between the first-type medical image and the second-type medical image.

2. The medical image processing apparatus according to claim 1, wherein the processing circuitry
associates the first-type feature point with the second-type feature point based on the corrected result of position adjustment, and
from among the pairs subjected to association based on the corrected result of position adjustment, presents, as candidates for correction in association, the pairs in which combination of the first-type feature point and the second-type feature point is different than the pairs in which the first-type feature point and the second-type feature point are associated based on result of position adjustment.

3. The medical image processing apparatus according to claim 2, wherein the processing circuitry
displays, in a display unit, the extracted first-type feature point, the extracted second-type feature point, and association information indicating the pairs subjected to association, and
after presenting the candidates for correction, displays, in the display unit, the association information equivalent to the presented candidates for correction in a distinguishable manner from the association information not corresponding to the candidates for correction.

4. The medical image processing apparatus according to claim 3, wherein the processing circuitry displays the association information in the display unit by displaying the first-type feature point and the second-type feature point, which corresponds to the first-type feature point, either one above other or side by side.

5. The medical image processing apparatus according to claim 4, wherein the processing circuitry
displays, in the display unit and in order based on positions of first-type feature points, the first-type feature point and the second-type feature point along with displaying the association information, and
associates, with the second-type feature point, only the first-type feature point lower in the order than the first-type feature point specified in the correction input.

6. The medical image processing apparatus according to claim 4, wherein the processing circuitry displays the association information in the display unit by enclosing the first-type feature point and the second-type feature point, which corresponds to the first-type feature point, in a frame.

7. The medical image processing apparatus according to claim 4, wherein the processing circuitry displays the association information in the display unit by joining the first-type feature point and the second-type feature point, which corresponds to the first-type feature point, by a line.

8. The medical image processing apparatus according to claim 3, wherein, after presenting the candidates for correction, the processing circuitry displays, in the display unit, only the association information equivalent to the presented candidates for correction.

9. The medical image processing apparatus according to claim 3, wherein, for each target body part of test subject to be examined, the processing circuitry displays the extracted first-type feature point, the extracted second-type feature point, and the association information in the display unit.

10. The medical image processing apparatus according to claim 9, wherein the processing circuitry
displays, in a tiled manner, the first-type medical image and the second-type medical image that have the target body part captured therein, and
displays the association information in the display unit by joining the first-type feature point, which is displayed in the first-type medical image, and the second-type feature point, which is displayed in the second-type medical image and which corresponds to the first-type feature point, by a line.

11. A medical image processing method implemented in a medical image processing apparatus, comprising:
obtaining a first-type medical image, and obtaining a second-type medical image which is taken at a different timing than the first-type medical image and which includes a region captured in the first-type medical image,
extracting a first-type feature point from the first-type medical image, and extracting a second-type feature point from the second-type medical image,
based on result of position adjustment between the first-type medical image and the second-type medical image, associating a first-type feature point and a second-type feature point captured in corresponding part in the first-type medical image and in the second-type medical image,
receiving a correction input that, from among pairs of the first-type feature point and the second-type feature point associated with each other, is meant for associating the first-type feature point with other second-type feature point,
calculating that includes
moving the other second-type feature point, which is specified in the correction input, to a position that is in the second-type medical image and that corresponds to the first-type feature point specified in the correction input, and
calculating position difference between pre-movement position and post-movement position of the other second-type feature point, and
correcting the result of position adjustment based on the position difference.

12. A memory medium having a computer program stored therein, wherein the computer program implements:
obtaining a first-type medical image, and obtaining a second-type medical image which is taken at a different timing than the first-type medical image and which includes a region captured in the first-type medical image,
extracting a first-type feature point from the first-type medical image, and extracting a second-type feature point from the second-type medical image,
based on result of position adjustment between the first-type medical image and the second-type medical image, associating a first-type feature point and a second-type feature point captured in corresponding part in the first-type medical image and in the second-type medical image,
receiving a correction input that, from among pairs of the first-type feature point and the second-type feature point associated with each other, is meant for associating the first-type feature point with other second-type feature point,
calculating that includes
moving the other second-type feature point, which is specified in the correction input, to a position that is in the second-type medical image and that corresponds to the first-type feature point specified in the correction input, and
calculating position difference between pre-movement position and post-movement position of the other second-type feature point, and
correcting the result of position adjustment based on the position difference.
